(19) United States
(12) Patent Application Publication
(10) Pub. No.: US 2020/0216813 A1
(43) Pub. Date: Jul. 9, 2020

(54) CELL REPROGRAMMING TO CARDIOMYOCYTES

(71) Applicant: Mogrify Limited, Cambridge (GB)

(72) Inventors: Julian Gough, Cambridge (GB); Owen Rackham, Cambridge (GB); Justin Cooper-White, Queensland (AU)

(73) Assignee: Mogrify Limited, Cambridge (GB)

(21) Appl. No.: 16/625,268

(22) PCT Filed: Jun. 21, 2018
(86) PCT No.: PCT/AU2018/050616

§ 371 Date: Dec. 20, 2019

(30) Foreign Application Priority Data
Jun. 21, 2017 (AU) 2017902384

(51) Int. Cl. C12N 5/077; C12N 15/85

(52) U.S. Cl. CPC C12N 5/0657; C12N 2510/00; C12N 2506/1307; C12N 15/85

(57) ABSTRACT

The present invention relates to methods and compositions, including kits for reprogramming a source cell to a cardiomyocyte, the method comprising increasing the protein expression of one or more transcription factors, or variants thereof, in the source cell, wherein the source cell is reprogrammed to exhibit at least one characteristic of a cardiomyocyte.

### RELATED APPLICATION

This application claims priority from Australian provisional application AU 2017902384, the entire contents of which are hereby incorporated by reference.

### FIELD OF THE INVENTION

The invention relates to methods and compositions for converting various cell types to cardiomyocytes.

### BACKGROUND OF THE INVENTION

Cell-based regenerative therapy requires the generation of specific cell types for replacing tissues damaged by injury, disease or age. Embryonic stem cells (ESC) have the potential to differentiate in every cell type from the (human) body and have therefore been extensively studied as a source for replacement therapy. However, ESC cannot be derived in a patient-specific fashion since they are established from cultured blastocysts. Therefore, immune rejection and ethical concerns are the main barriers that prevent the transfer of the ESC technology, and in particular of human ESC technology, to clinical applications.

Cell-replacement therapies have the potential to rapidly generate a variety of therapeutically important cell types directly from one's own easily accessible tissues, such as skin or blood. Such immunologically-matched cells would also pose less risk for rejection after transplantation. Moreover, these cells would manifest less tumorigenicity since they are terminally differentiated.

Trans-differentiation, the process of converting from one cell type to another without going through a pluripotent state, may have great promise for regenerative medicine but has yet to be reliably applied. Although it may be possible to switch the phenotype of one somatic cell type to another, the elements required for conversion are difficult to identify and in most instances unknown. The identification of factors to directly reprogram the identity of cell types is currently limited by, amongst other things, the cost of exhaustive experimental testing of plausible sets of factors, an approach that is inefficient and unscalable.

The mammalian heart lacks significant regenerative capacity. In vitro generation of cardiomyocytes in quantities sufficient for transplantation would therefore greatly assist the treatment of heart disease.

There is a need for a new and/or improved method for generating cardiomyocytes or cells displaying characteristics of a cardiomyocyte cell, both in vitro and in vivo.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

The present invention provides a method for reprogramming a source cell, the method comprising increasing the protein expression of one or more transcription factors, or variants thereof, in the source cell, wherein the source cell is reprogrammed to exhibit at least one characteristic of a cardiomyocyte, wherein:
the source cell is selected from the group consisting of cardiac fibroblasts and mesenchymal stem cells,
the transcription factors are one or more of the transcription factors listed in Tables 1, 2 and 3.

The present invention provides a method of generating a cell exhibiting at least one characteristic of a cardiomyocyte from a source cell, the method comprising:
increasing the amount of one or more transcription factors, or variants thereof, in the source cell; and
culturing the source cell for a sufficient time and under conditions to allow differentiation to a cardiomyocyte; thereby generating the cell exhibiting at least one characteristic of a cardiomyocyte from a source cell, wherein:
the source cell is selected from the group consisting of cardiac fibroblasts, and mesenchymal stem cells and
the transcription factors are one or more of those listed in Table 1, 2 and 3.

The present invention also provides a method for reprogramming a cardiac fibroblast or mesenchymal stem cell, the method comprising increasing the protein expression of one or more of the transcription factors in Tables 1, 2 or 3, or variants thereof, in the source cell, wherein the source is reprogrammed to exhibit at least one characteristic of cardiomyocyte.

The present invention provides a method for reprogramming a source cell to a cell that exhibits at least one characteristic of a cardiomyocyte comprising: i) providing a source cell, or a cell population comprising a source cell; ii) contacting said source cell with one or more agents that activate or increase the expression of one or more transcription factors; and iii) culturing said cell or cell population, and optionally monitoring the cell or cell population for at least one characteristic of a cardiomyocyte cell, wherein:
the source cell is selected from the group consisting of cardiac fibroblasts and mesenchymal stem cells
the transcription factors are one or more of those listed in Tables 1, 2 and 3.

In any method of the present invention, the source cell is a fibroblast, including a cardiac fibroblast, and a fetal cardiac fibroblast, and the target cell is a cardiomyocyte and the transcription factors are any one or more transcription factors listed in Tables 1 and 3.

In a preferred embodiment, any one or more, or all of the transcription factors in a single row of Table 1 may be used. For example, the transcription factors are any one or more of:

(a) BMP10, GATA6, TBX5, ANKRD1, HAND2, PPARGC1A, NKX2-5 and GATA4;

(b) TBX5, GATA6, ANKRD1, HAND2, HAND1, PPARGC1A, NKX2-5 and GATA4;

(c) GATA6, ANKRD1, HAND1, PPARGC1A, S100A1, NKX2-5 and GATA4;

(d) FHL2, ANKRD1, HAND1, PPARGC1A, S100A1, MYOCD, NKX2-5 and GATA4;

(e) TBX20, ANKRD1, HAND1, HAND2, PPARGC1A, MYOCD, NKX2-5 and GATA4;

(f) ANKRD1, HAND1, HAND2, PPARGC1A, S100A1, MYOCD, NKX2-5 and GATA4;

(g) HAND2, HAND1, PPARGC1A, S100A1, MYOCD, KLHL31, NKX2-5 and GATA4;

(h) HAND1, PPARGC1A, S100A1, MYOCD, KLHL31, NKX2-5, GATA4 and TCF21;

(i) SMYD1, PPARGC1A, S100A1, ABRA, MYOCD, KLHL31, NKX2-5 and GATA4;

(j) PPARGC1A, S100A1, ABRA, MYOCD, KLHL31, NKX2-5, GATA4 and TCF21

(k) S100A1, ABRA, MYOCD, KLHL31, NKX2-5, GATA4 and TCF21;

(l) E2F8, ABRA, MYOCD, KLHL31, NKX2-5, GATA4 and TCF21;

(m) ABRA, MYOCD, KLHL31, NKX2-5, GATA4 and TCF21;

(n) MYOCD, KLHL31, NKX2-5, GATA4 and TCF21;

(o) IGF2, KLHL31, NKX2-5, GATA4 and TCF21; or

(p) KLHL31, NKX2-5, GATA4 and TCF21.

In (a) to (p) immediately above, all of the transcription factors listed may be used.

Alternatively, the transcription factors may be the combination of transcription factors listed as follows:

### (a) HAND1, TBX5, GATA6, ANKRD1, HAND2, PPARGC1A, NKX2-5 and GATA4;

### (b) BMP10, HAND1, GATA6, ANKRD1, HAND2, PPARGC1A, NKX2-5 and GATA4;

### (c) BMP10, TBX5, FHL2, ANKRD1, HAND2, PPARGC1A, NKX2-5 and GATA4;

### (d) BMP10, TBX5, GATA6, HAND1, HAND2, PPARGC1A, NKX2-5 and GATA4;

### (e) BMP10, TBX5, GATA6, ANKRD1, HAND1, PPARGC1A, NKX2-5 and GATA4;

### (f) BMP10, TBX5, GATA6, ANKRD1, HAND2, HAND1, NKX2-5 and GATA4;

### (g) BMP10, TBX5, GATA6, ANKRD1, HAND2, PPARGC1A, HAND1 and GATA4; or

### (h) BMP10, TBX5, GATA6, ANKRD1, HAND2, PPARGC1A, NKX2-5 and HAND1.

Preferably, the cardiac fibroblast is a human cardiac fibroblast. The cardiac fibroblast may be an interstitial fibroblast, or derived from endocardium or epicardium. The cardiac fibroblast may be a fetial cardiac fibroblast.

In any aspect of the invention the cardiomyocyte cell may be a fetal cardiomyocyte cell.

In any method of the invention described herein, the source cell is a mesenchymal stem cell, and the target cell is a cardiomyocyte and the transcription factors are any one or more of the transcription factors listed in Table 2.

In a preferred embodiment, any one or more, or all of the transcription factors listed in a single row of Table 2 may be used. Alternatively, the transcription factors may be the combination of the transcription factors listed as follows:

### (a) ANKRD1, TBX5, GATA6, FHL2, NKX2-5, HAND2, GATA4 and PPARGC1A;

### (b) BMP10, ANKRD1, GATA6, FHL2, NKX2-5, HAND2, GATA4 and PPARGC1A;

### (c) BMP10, TBX5, ANKRD1, FHL2, NKX2-5, HAND2, GATA4 and PPARGC1A;

### (d) BMP10, TBX5, GATA6, ANKRD1, NKX2-5, HAND2, GATA4 and PPARGC1A;

### (e) BMP10, TBX5, GATA6, FHL2, HAND1, HAND2, GATA4 and PPARGC1A;

### (f) BMP10, TBX5, GATA6, FHL2, NKX2-5, HAND1, GATA4 and PPARGC1A;

### (g) BMP10, TBX5, GATA6, FHL2, NKX2-5, HAND2, ANKRD1 and PPARGC1A; or

### (h) BMP10, TBX5, GATA6, FHL2, NKX2-5, HAND2, GATA4 and ANKRD1.

Preferably the mesenchymal stem cell is a human mesenchymal stem cell.

Preferably, the at least one characteristic of the cardiomyocyte cell is up-regulation of any one or more target cell markers and/or change in cell morphology. Relevant markers are described herein and known to those in the art. Exemplary markers for fetal cardiomyocytes: MEF2C, MYH6, ACTN1, CDH2 and GJA1. The marker NCX-1 is an indicator that the target cell exhibits the capacity to contract.

Typically, conditions suitable for target cell differentiation include culturing the cells for a sufficient time and in a suitable medium. A sufficient time of culturing may be at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 days. A suitable medium may be one shown in Table 4.

In any method described herein, the method may further include the step of expanding the cells exhibiting at least one characteristic of a target cell type to increase the proportion of cells in the population exhibiting at least one characteristic of a cardiomyocyte cell. The step of expanding the cells may be in culture for a sufficient time and under conditions for generating a population of cells as described below.

In any method described herein, the method may further include the step of administering the cells, or cell population including a cell, exhibiting at least one characteristic of a cardiomyocyte cell, to an individual.

The present invention also provides a use of a cell, or cell population including a cell produced according to the instant invention, exhibiting at least one characteristic of a cardiomyocyte cell, in the manufacture of a medicament for the treatment of a disease of the heart. For example, the reprogrammed cells of the instant invention may be introduced (transplanted) to a failing or infarcted heart (e.g., after a heart attack) in order to improve cardiac function.

The present invention also provides a cell exhibiting at least one characteristic of a cardiomyocyte cell produced by a method as described herein. Preferably, the cardiomyocyte cell is a fetal cardiomyocyte cell. Typically, the cardiomyocyte cell expresses the marker NCX-1 and is contractile.

The present invention also provides a population of cells, wherein at least 5% of cells exhibit at least one characteristic of a cardiomyocyte cell and those cells are produced by a method as described herein. Preferably, at least 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% or 100% of the cells in the population exhibit at least one characteristic of a cardiomyocyte cell.

The present invention also relates to kits for producing a cell exhibiting at least one characteristic of a cardiomyocyte cell as disclose herein. In some embodiments, a kit comprises one or more nucleic acids having one or more nucleic acid sequences encoding a transcription factor described herein or variant thereof. Preferably, the kit can be used to produce a cell exhibiting at least one characteristic of a cardiomyocyte cell. Preferably, the kit can be used with a source cell that is a cardiac fibroblast or an mesenchymal stem cell. In some embodiments, the kit further comprises instructions for reprogramming a source cell to a cell exhibiting at least one characteristic of a cardiomyocyte cell according to the methods as disclosed herein. Preferably, the present invention provides a kit when used in a method of the invention described herein.

The present invention relates to a composition comprising at least one cardiomyocyte cell and at least one agent which increases the protein expression of one or more transcription factors in the target cell. Further, the transcription factor may be any one described herein. Preferably, the transcription factors are as described in any one of Tables 1, 2 or 3.

The present invention provides a method for reprogramming a cardiac fibroblast cell, the method comprising increasing the protein expression of any one or more of the transcription factors listed in Table 1, or all of the transcription factors listed in a single row of Table 1, or alternatively the transcription factors selected from:

### (a) HAND1, TBX5, GATA6, ANKRD1, HAND2, PPARGC1A, NKX2-5 and GATA4;

### (b) BMP10, HAND1, GATA6, ANKRD1, HAND2, PPARGC1A, NKX2-5 and GATA4;

### (c) BMP10, TBX5, FHL2, ANKRD1, HAND2, PPARGC1A, NKX2-5 and GATA4;

### (d) BMP10, TBX5, GATA6, HAND1, HAND2, PPARGC1A, NKX2-5 and GATA4;

### (e) BMP10, TBX5, GATA6, ANKRD1, HAND1, PPARGC1A, NKX2-5 and GATA4;

### (f) BMP10, TBX5, GATA6, ANKRD1, HAND2, HAND1, NKX2-5 and GATA4;

### (g) BMP10, TBX5, GATA6, ANKRD1, HAND2, PPARGC1A, HAND1 and GATA4; or

### (h) BMP10, TBX5, GATA6, ANKRD1, HAND2, PPARGC1A, NKX2-5 and HAND1.

or variants thereof, in the fibroblast cell, wherein the fibroblast cell is reprogrammed to exhibit at least one characteristic of a cardiomyocyte cell.

The present invention provides a method of generating a cell exhibiting at least one characteristic of a cardiomyocyte cell from a cardiac fibroblast cell, the method comprising:
increasing the amount of any one or more of BMP10, GATA6, TBX5, ANKRD1, HAND2, PPARGC1A, NKX2-5 and GATA4, or variants thereof, in the fibroblast cell; and
culturing the fibroblast cell for a sufficient time and under conditions for cardiomyocyte cell differentiation; thereby generating the cell exhibiting at least one characteristic of a cardiomyocyte cell from a fibroblast cell.

The present invention also relates to kits for producing a cell exhibiting at least one characteristic of a cardiomyocyte cell as disclose herein. In some embodiments, a kit comprises any one or more of (i) a nucleic acid sequence encoding a BMP10 polypeptide or variant thereof; and (ii) a nucleic acid sequence encoding a GATA6 polypeptide or variant thereof; and (iii) a nucleic acid sequence encoding a TBX5 polypeptide or variant thereof, and (iv) a nucleic acid sequence encoding a ANKRD1 polypeptide or variant thereof, and (v) a nucleic acid sequence encoding a HAND2 polypeptide or variant thereof, and (vi) a nucleic acid sequence encoding a PPARGC1A polypeptide or variant thereof, and (vii) a nucleic acid sequence encoding a NKX2-5 polypeptide or variant thereof, and (viii) a nucleic acid sequence encoding a GATA4 polypeptide or variant thereof.

In some embodiments, the kit further comprises instructions for reprogramming a cardiac fibroblast cell to a cell exhibiting at least one characteristic of a cardiomyocyte cell according to the methods as disclosed herein. Preferably, the present invention provides a kit when used in a method of the invention described herein.

The present invention relates to a composition comprising at least one cardiac fibroblast cell and at least one agent which increases the protein expression of any one or more of BMP10, GATA6, TBX5, ANKRD1, HAND2, PPARGC1A, NKX2-5 and GATA4 in the fibroblast cell.

In any aspect of the invention, the at least one characteristic of a cardiomyocyte cell is the ability to contract. In other words, the at least one characteristic of a cardiomyocyte cell is the capacity to beat.

Typically, the protein expression, or amount, of a transcription factor as described herein is increased by contacting the cell with an agent which increases the expression of the transcription factor. Preferably, the agent is selected from the group consisting of: a nucleotide sequence, a protein, an aptamer and small molecule, ribosome, RNAi agent and peptide-nucleic acid (PNA) and analogues or variants thereof. In some embodiments, the agent is exogenous. The present invention also contemplates the use of a transcriptional activation system (e.g., a gRNA for use in a gene activation system such as CRISPR/Cas9 or TALEN) for increasing the expression of the one or more transcription factors.

Typically, the protein expression, or amount, of a transcription factor as described herein is increased by introducing at least one nucleic acid comprising a nucleotide sequence encoding a transcription factor, or encoding a functional fragment thereof, in the cell. Preferably, the nucleotide sequence encoding a transcription factor is at least 70%, 80%, 85%, 90%, 91%, 92%, 93%, 94%, 95%, 96%, 97%, 98%, or 99% identical to a sequence with an accession number listed in Table 3

In a preferred embodiment of the invention, the nucleic acid sequence encoding a transcription factor protein is introduced into a cell by a plasmid. One or more nucleic acids encoding one or more transcription factors may be used. Therefore, it is apparent that one or more plasmids may be used for the purpose of increasing the expression or amount of the required one or more transcription factors. In other words, the nucleic acid sequences may be in or on a single plasmid, or provided to the source cell in two or more plasmids.

In any embodiment of the present invention, the plasmid containing the nucleic acid encoding the one or more transcription factors for use according to the invention may be an episomal plasmid.

In any embodiment of the present invention, a detectable marker may also be introduced into the source cell to identify when the source cell has been reprogrammed to exhibit at least one characteristic of a cardiomyocyte cell. The detectable marker may be a fluorescent reporter operably linked to a reporter or enhancer sequence from the SLC8A1 gene.

Preferably, the nucleic acid further includes a heterologous promoter. Preferably, the nucleic acid is in a vector, such as a viral vector or a non-viral vector. Preferably, the vector is a viral vector comprising a genome that does not integrate into the host cell genome. The viral vector may be a retroviral vector or a lentiviral vector.

Any method as described herein may have one or more, or all, steps performed in vitro, ex vivo or in vivo.

As used herein, except where the context requires otherwise, the term “comprise” and variations of the term, such as “comprising”, “comprises” and “comprised”, are not intended to exclude further additives, components, integers or steps.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1. Relative expression levels of the individual factors ANKRD1, BMP10, GATA6, TBX5, HAND1, PPARGC1A, NKX2.5 and GATA4, shown as a function of time, following transfection of cardiac fibroblasts with an episomal plasmid encoding each factor.

FIG. 2. Relative expression levels of myh6 and cTnT as a function of time following transfection of cardiac fibroblasts with a plasmid encoding the transcription factors identified in FIG. 1 (relative to negative control (hCFCs)).

FIG. 3. A. FACS plot of GFP positive cells. B. LHS: NCX-1-GFP+ cells in total population at day 8; Middle: NCX-1-GFP+ cells at day 8 (sorted and reseeded); RHS: NCX-1-GFP+ cells at day 14 (post sorting and reseeding at day 8).

FIG. 4. Relative expression level of cardiac markers, including myh6, cTnT, and NCX (to negative control hFCFs) of NCX-1-GFP reporter cell line (without sorting) after transfection with the nucleic acids encoding the transcription factors.

### DETAILED DESCRIPTION OF THE EMBODIMENTS

It will be understood that the invention disclosed and defined in this specification extends to all alternative combinations of two or more of the individual features mentioned or evident from the text or drawings. All of these different combinations constitute various alternative aspects of the invention.

Reference will now be made in detail to certain embodiments of the invention. While the invention will be described in conjunction with the embodiments, it will be understood that the intention is not to limit the invention to those embodiments. On the contrary, the invention is intended to cover all alternatives, modifications, and equivalents, which may be included within the scope of the present invention as defined by the claims.

One skilled in the art will recognize many methods and materials similar or equivalent to those described herein, which could be used in the practice of the present invention. The present invention is in no way limited to the methods and materials described. It will be understood that the invention disclosed and defined in this specification extends to all alternative combinations of two or more of the individual features mentioned or evident from the text or drawings. All of these different combinations constitute various alternative aspects of the invention.

For purposes of interpreting this specification, terms used in the singular will also include the plural and vice versa.

The process of reprogramming a cell alters the type of progeny a cell can produce and includes the distinct processes of forward programming and transdifferentiation. In some embodiments, forward programming of multipotent cells or pluripotent cells provides cells exhibiting at least one characteristic of a cell type having a more differentiated phenotype than the multipotent cell or pluripotent cell. In other embodiments, transdifferentiation of one somatic cell provides a cell exhibiting at least one characteristic of another somatic cell type.

The present invention provides compositions and methods for direct reprogramming or transdifferentiation of source cells to target cells, without the source cell becoming an induced pluripotent stem cell (iPS) intermediately prior to becoming a target cell. In comparison to iPS cell technology, transdifferentiation is highly efficient and poses a very low risk of teratoma formation for downstream applications. Moreover, transdifferentiation can be used in vivo for the direct conversion of one cell type into another, whereas iPS cell technology cannot.

The present invention is particularly directed towards the conversion or transdifferentiation of source cells into fetal cardiomyocytes. These fetal cardiomyocytes may have utility in a wide range of applications, including for the generation of cardiac cells in quantities sufficient for transplantation, including to assist in the treatment of heart disease. In particular, fetal cardiomyocytes produced according to the present invention may be introduced (transplanted) to a failing or infarcted heart (e.g., after a heart attack) in order to improve cardiac function. A benefit of using fetal cardiomyocytes is that these cells have the ability to proliferate and can electrically and mechanically connect to the host cardiac tissue.

Cells produced according to the present invention may also find utility in a variety of other clinical applications, including for the testing of drugs requiring regulatory approval and for disease modelling.

A particular advantage of the present invention, is that it enables the skilled person to produce large amount of fetal cardiomyocytes. While the conversion of certain cells to cardiomyocytes is reported in the literature, these conversions are very slow and produce a small proportion of converted cells. This means that the methods of the prior art are limited in their application in a clinical context. In contrast, the methods of the present invention result in a much higher conversion rate than the methods of the prior art.

Moreover, certain methods of the present invention enable the production of fetal cardiomyocytes from a source cell. In other words, what can be produced is a precursor to a mature, or fully developed cardiomyocyte. This has the further advantage of producing a cell population that can be more successfully transplanted into a recipient without causing scarring. Moreover, fetal cardiomyocytes have a greater capacity to regenerate and proliferate as compared to adult or mature cardiomyocytes.

As used herein, a fetal cardiomyocte is a reference to any cell that has the characteristics of a fetal cardiomyocyte. A cell may be defined as having the characteristics of a fetal cardiomyocyte based on one or more markers, including cell surface markers, gene expression levels or production of macromolecules. The characteristic may also be one or more morphological traits.

A source cell may be any cell type described herein, including a somatic cell or a diseased cell. The somatic cell may be an adult cell or a cell derived from an adult which displays one or more detectable characteristics of an adult or non-embryonic cell. The diseased cell may be a cell displaying one or more detectable characteristics of a disease or condition.

As used herein, “transdifferentiation” refers to a method of reprogramming a somatic cell of one type (or having the characteristics of one type of somatic cell), such that the morphological and functional properties of the cell and converted into that of another cell type (without undergoing an intermediate pluripotent state or progenitor cell type). The term transdifferentiation may be used interchangeably with the terms “lineage reprogramming” or “cell reprogramming” or “cell conversion”.

A source cell may be any cell type described herein, including a somatic cell or a diseased cell. The somatic cell may be an adult cell or a cell derived from an adult which displays one or more detectable characteristics of an adult or non-embryonic cell. The diseased cell may be a cell displaying one or more detectable characteristics of a disease or condition. Examples of source cells include a fibroblast (including a cardiac fibroblast) and mesenchymal stem cells.

As used herein, the term “somatic cell” refers to any cell forming the body of an organism, as opposed to germline cells. In mammals, germline cells (also known as “gametes”) are the spermatozoa and ova which fuse during fertilization to produce a cell called a zygote, from which the entire mammalian embryo develops. Every other cell type in the mammalian body—apart from the sperm and ova, the cells from which they are made (gametocytes) and undifferentiated stem cells—is a somatic cell: internal organs, skin, bones, blood, and connective tissue are all made up of somatic cells. In some embodiments the somatic cell is a “non-embryonic somatic cell”, by which is meant a somatic cell that is not present in or obtained from an embryo and does not result from proliferation of such a cell in vitro. In some embodiments the somatic cell is an “adult somatic cell”, by which is meant a cell that is present in or obtained from an organism other than an embryo or a fetus or results from proliferation of such a cell in vitro. The somatic cells may be immortalized to provide an unlimited supply of cells, for example, by increasing the level of telomerase reverse transcriptase (TERT). For example, the level of TERT can be increased by increasing the transcription of TERT from the endogenous gene, or by introducing a transgene through any gene delivery method or system.

Unless otherwise indicated the methods for reprogramming somatic cells can be performed both in vivo and in vitro (where in vivo is practiced when somatic cells are present within a subject, and where in vitro is practiced using isolated somatic cells maintained in culture).

Differentiated somatic cells, including cells from a fetal, newborn, juvenile or adult primate, including human, individual, are suitable source cells in the methods of the invention. Suitable somatic cells include, but are not limited to fibroblast cells, including cardiac fibroblast cells. Suitable somatic cells are receptive, or can be made receptive using methods generally known in the scientific literature, to uptake of transcription factors including genetic material encoding the transcription factors. Uptake-enhancing methods can vary depending on the cell type and expression system. Exemplary conditions used to prepare receptive somatic cells having suitable transduction efficiency are well-known by those of ordinary skill in the art. The starting somatic cells can have a doubling time of about twenty-four hours.

The term “isolated cell” as used herein refers to a cell that has been removed from an organism in which it was originally found or a descendant of such a cell. Optionally the cell has been cultured in vitro, e.g., in the presence of other cells. Optionally the cell is later introduced into a second organism or re-introduced into the organism from which it (or the cell from which it is descended) was isolated.

The term “isolated population” with respect to an isolated population of cells as used herein, refers to a population of cells that has been removed and separated from a mixed or heterogeneous population of cells. In some embodiments, an isolated population is a substantially pure population of cells as compared to the heterogeneous population from which the cells were isolated or enriched from.

The term “substantially pure”, with respect to a particular cell population, refers to a population of cells that is at least about 75%, preferably at least about 85%, more preferably at least about 90%, and most preferably at least about 95% pure, with respect to the cells making up a total cell population. Recast, the terms “substantially pure” or “essentially purified”, with regard to a population of target cells, i.e. cells at exhibit at least one characteristic of a cardiomyocyte cell, refers to a population of cells that contain fewer than about 20%, more preferably fewer than about 15%, 10%, 8%, 7%, most preferably fewer than about 5%, 4%, 3%, 2%, 1%, or less than 1%, of cells that are not target cells or their progeny as defined by the terms herein.

The skilled person will also be familiar with means to distinguish the characteristics of source cells from those of cardiomyocytes (in other words, to test for the loss of source cell phenotype). For example, as provided in the examples herein, suitable source cells for the production of fetal cardiomyocytes include fetal cardiac fibroblasts and mesenchymal stem cells. The skilled person will be able to readily distinguish between characteristics of a cardiac fibroblast and a cardiomyocyte, for example: cardiac fibroblasts are typically positive for vimentin, periostin, fibroblast-specific protein (FSP), Thy-1 (CD90), and alpha-smooth muscle actin (alpha SMA).

The skilled person will also be readily able to distinguish between a mesenchymal stem cell and a cardiomyocyte including a fetal cardiomyocyte. For example, based on the minimal criterial of the International Society of Cellular Therapy (ISCT), human mesenchymal stem cells can be identified by adherence to plastic and expression of cell surface markers CD29, CD44, D73 (SH3), CD90, CD49a-f, CD51, CD105 (SH2), CD106, CD166 and Stro-1 and the lack of expression of CD45, CD34, CD14 or CD11b, CD79a or CD19 and HLA-DR surface molecules. Other markers which may be expressed on these cells include CD146, CD140b, Stro-3 and Stro-4. The skilled person will be able to determine conversion from mesenchymal stem cells to cardiomyocytes by observing changes in expression of these markers.

A source cell is determined to be converted to a cardiomyocyte when it displays at least one characteristic of a cardiomyocyte. For example, a fibroblast will be identified as being converted to a cardiomyocyte when a fibroblast, treated according to a method of the present invention, displays at least one characteristic of cardiomyocyte. Moreover, a human fetal fibroblast, including a human fetal cardiac fibroblast, will be identified as being converted to a fetal cardiomyocyte when it displays at least one characteristic of a fetal cardiomyocyte.

The one or more characteristics of a cardiomyocyte including a fetal cardiomyocyte include up-regulation of any one or more cardiomyocyte markers and/or changes in cell morphology. Typically, a cell that is converted to a cardiomyocyte will display 1, 2, 3, 4, 5, 6, 7, 8 or more characteristics of the cardiomyocyte.

In any embodiment of the present invention, a protein marker which is characteristic of a cardiomyocyte includes Myosin heavy chain, a isoform (MHC-α), cardiac troponin (cTnT), MEF2C, MYH6, ACTN1, CDH2, GJA1 and NKX2.5. Preferably, the cardiomyocyte cell expresses the marker NCX-1 which indicates it has the ability to contract.

Fetal cardiomyocytes also have the ability to beat without external stimulation, while mature (adult) cardiomyoctes require pacing from an external source (such as electrical stimulation or drugs in vitro, or pacemaker cells or purkinje cells in vivo).

Additional markers which can be used to determine whether a source cell has been reprogrammed or converted into a cardiomyocyte will be known to the skilled person. Examples of suitable markers are disclosed for example in Xu et al., (2009) Stem Cells, 27: 2163-2174 and in Robertson et al., (2013) Stem Cells, 31: doi:10.1002/stem.1331, the entire contents of which are herein incorporated in their entirety.

The skilled person will be familiar with methods for determining conversion of a cardiac fibroblast or mesenchymal stem cell to a cardiomyocyte by observing changes in cell morphology. For example, adult cardiomyocytes are rod-shaped cells that display well organised perpendicular striation of sarcomeric actinin and longitudinally organised mitochondria. Adult cardiomyocytes can also be distinguished from fetal cardiomyocytes. Adult cardiomyocytes are larger than fetal cardiomyocytes, with multiple nuclei and sarcoplasmic reticulum, large sarcomeric area, and large numbers of mitochondria. Fetal cardiomyocytes appear more rounded or oblate in shape and show lower levels of striation and organised sarcomeric actinin and randomly organised mitochondria.

In any aspect of the invention, the cardiomyocyte characteristic may be determined by analysis of cell morphology, gene expression profiles, activity assay, protein expression profile, surface marker profile, differentiation ability or a combination thereof. Examples of characteristics or markers include those that are described herein and those known to the skilled person.

The transcription factors that may be used to convert a cardiac fibroblast to a cell that exhibits at least one characteristic of a cardiomyocyte are shown below in Table 1.

Any one or more of the transcription factors as shown in each row of Table 1 may be used to transdifferentiate a cardiac fibroblast into a cardiomyocyte. For example, the protein expression or amount of any one, two, three, four, five, six, seven or all eight transcription factors as shown in each row may be used for the purposes of the present invention.

TABLE 1
Transcription factors for reprogramming of cardiac fibroblasts to cardiomyocytes

| | | | | | | | | % |
|---|---|---|---|---|---|---|---|---|
| TF1 | TF2 | TF3 | TF4 | TFS | TFS | TF7 | TFS | coverage |
| BMP10 | TBX5 | GATA6 | ANKRD1 | HAND2 | PPARGC1A | NKX2-5 | GATA4 | 93.52718 |
| TBX5 | GATA6 | ANKRD1 | HAND2 | HAND1 | PPARGC1A | NKX2-5 | GATA4 | 33.52718 |
| GATA6 | ANKRD1 | HAND2 | HAND1 | PPARGC1A | S100A1 | NKX2-5 | GATA4 | 92.98370 |
| FHL2 | ANKRD1 | HAND2 | PPARGC1A | S100A1 | MYOCD | NKX2-5 | GATA4 | 70.56524 |
| TBX20 | ANKRD1 | HAND2 | PPARGC1A | S100A1 | MYOCD | NKX2-5 | GATA4 | 70.49730 |
| ANKRD1 | HAND2 | HAND1 | PPARGC1A | S100A1 | MYOCD | NKX2-5 | GATA4 | 70.43730 |
| HAND2 | HAND1 | PPARGC1A | S100A1 | MYOCD | KLHL31 | NKX2-5 | GATA4 | 70.35143 |
| HAND1 | PPARGC1A | S100A1 | MYOCD | KLHL31 | NKX2-5 | GATA4 | TCF21 | 70.29350 |
| SMYD1 | PPARGC1A | S100A1 | ABRA | MYOCD | KLHL31 | NKX2-5 | GATA4 | 45.83699 |
| PPARGC1A | S100A1 | ABRA | MYOCD | KLHL31 | NKX2-5 | GATA4 | TCF21 | 45.97285 |
| S100A1 | ABRA | MYOCD | KLHL31 | NKX2-5 | GATA4 | TCF21 | NA | 45.02177 |
| E2F8 | ABRA | MYOCD | KLHL31 | NKX2-5 | GATA4 | TCF21 | NA | 44.88590 |
| ABRA | MYOCD | KLHL31 | NKX2-5 | GATA4 | TCF21 | NA | NA | 44.88590 |
| MYOCD | KLHL31 | NKX2-5 | GATA4 | TCF21 | NA | NA | NA | 44.88590 |
| IGF2 | KLHL31 | NKX2-5 | GATA4 | TCF21 | NA | NA | NA | 44.47829 |
| KLHL31 | NKX2-5 | GATA4 | TCF21 | NA | NA | NA | NA | 44.47829 |
| NKX2-5 | GATA4 | TCF21 | NA | NA | NA | NA | NA | 44.47829 |
| GATA4 | TCF21 | NA | NA | NA | NA | NA | NA | 30.55167 |
| LBH | TCF21 | NA | NA | NA | NA | NA | NA | 1.203872 |
| TCF21 | NA | NA | NA | NA | NA | NA | NA | 1.135937 |

As used herein, percentage coverage (% coverage) refers to the percentage of genes that are directly regulated by the listed transcription factors and for which expression is predicted to be altered between the source cell and the target cell type. For example, the transcription factors shown in row 1 of Table 1 directly regulate the expression of 93.527% of those genes whose expression is being targeted in order to convert the source cell to the target cell.

The inventors have also found that in addition to the above preferred groupings of transcription factors, there are some transcription factors which can be readily substituted for others. For example, in the context of the preferred group of transcription factors as shown in row 1 of Table 1, BMP10, TBX5, ANKRD1, HAND2, PPARGC1, NKX2-5 and GATA4, the inventors have found that any of these transcription factors can be replaced with the transcription factor HAND1. In other words, if it is not possible to increase the protein expression or amount of any of the given transcription factors BMP10, TBX5, ANKRD1, HAND2, PPARGC1, NKX2-5 and GATA4, (for example, if there is no nucleic acid construct available for increasing expression directly), then it is possible to seek to increase the expression of HAND1 instead. Put in other words, the transcription factor HAND1 can substitute for any of BMP10, TBX5, ANKRD1, HAND2, PPARGC1, NKX2-5 and GATA4, when seeking to transdifferentiate a cardiac fibroblast to a cardiomyocyte, according to the present methods.

In a similar context, the transcription factor GATA6 can be replaced with the transcription factor FHL2. As such, when using the factors BMP10, TBX5, ANKRD1, HAND2, PPARGC1, NKX2-5 and GATA4, (including wherein any one of these is substituted with HAND1), it is also possible to use either GATA6 or FHL2 for the purpose of converting a cardiac fibroblast to a cardiomyocte.

The transcription factors that may be used to convert a mesenchymal stem cell to a cell that exhibits at least one characteristic of a cardiomyocyte are shown below in Table 2.

Any one or more of the transcription factors as shown in each row of Table 2 may be used to convert a mesenchymal stem cell into a cardiomyocyte. For example, the protein expression or amount of any one, two, three, four, five, six, seven or all eight transcription factors as shown in each row may be used for the purposes of the present invention

TABLE 2
Transcription factors for conversion of mesenchymal stem cells to cardiomyocytes

| | | | | | | | | % |
|---|---|---|---|---|---|---|---|---|
| TF1 | TF2 | TF3 | TF4 | TF5 | TF6 | TF7 | TF8 | coverage |
| BMP10 | TBX5 | GATA6 | FHL2 | NKX2-5 | HAND2 | GATA4 | PPARGC1A | 90.72087 |
| TBX5 | GATA6 | FHL2 | NKX2-5 | ANKRD1 | HAND2 | GATA4 | PPARGC1A | 90.72087 |
| GATA6 | FHL2 | NKX2-5 | ANKRD1 | HAND2 | HAND1 | GATA4 | PPARGC1A | 90.14489 |
| FHL2 | TBX20 | NKX2-5 | ANKRD1 | HAND2 | GATA4 | PPARGC1A | MYOCD | 71.44751 |
| TBX20 | NKX2-5 | ANKRD1 | HAND2 | HAND1 | GATA4 | PPARGC1A | MYOCD | 71.35890 |
| NKX2-5 | ANKRD1 | HAND2 | HAND1 | GATA4 | PPARGC1A | S100A1 | MYOCD | 71.35890 |
| ANKRD1 | HAND2 | HAND1 | GATA4 | PPARGC1A | S100A1 | MYOCD | KLHL31 | 68.21313 |
| HAND2 | HAND1 | GATA4 | PPARGC1A | S100A1 | ABRA | MYOCD | KLHL31 | 68.16882 |
| HAND1 | GATA4 | PPARGC1A | S100A1 | ABRA | MYOCD | KLHL31 | TCF21 | 68.12452 |
| GATA4 | PPARGC1A | S100A1 | ABRA | MYOCD | KLHL31 | TCF21 | NA | 34.62874 |
| SMYD1 | PPARGC1A | S100A1 | ABRA | MYOCD | KLHL31 | TCF21 | NA | 4.5002.6 |
| PPARGC1A | S100A1 | ABRA | MYOCD | KLHL31 | TCF21 | NA | NA | 4.50026 |
| S100A1 | ABRA | MYOCD | KLHL31 | TCF21 | NA | NA | NA | 2.86092 |
| E2F8 | ABRA | MYOCD | KLHL31 | TCF21 | NA | NA | NA | 2.46216 |
| ABRA | MYOCD | KLHL31 | TCF21 | NA | NA | NA | NA | 2.46216 |
| MYOCD | KLHL31 | TCF21 | NA | NA | NA | NA | NA | 2.28493 |
| IGF2 | KLHL31 | TCF21 | LBH | NA | NA | NA | NA | 1.62033 |

The inventors have also found, that in addition to the above preferred groupings of transcription factors as shown in Table 2, there are some transcription factors or protein factors which can be readily substituted for others. For example, in the context of the preferred group of transcription factors as shown in row 1 of Table 2, BMP10, TBX5, GATA6, FHL2, NKX2-5, HAND2, GATA4 and PPARGC1A, the inventors have found that any of the transcription factors BMP10, TBX5, GATA6, FHL2, GATA4 and PPARGC1A can be replaced with the transcription ANKRD1. In other words, if it is not possible to increase the protein expression or amount of any of the given transcription factors BMP10, TBX5, GATA6, FHL2, GATA4 and PPARGC1A (for example, if there is no nucleic acid construct available for increasing expression directly), then it is possible to seek to increase the expression of ANKRD1 instead. Put in other words, the transcription factor ANKRD1 can substitute for any of BMP10, TBX5, GATA6, FHL2, GATA4 and PPARGC1A when seeking to transdifferentiate a mesenchymal stem cell to a fetal cardiomyocyte, according to the present methods.

Still further, the inventors have found that in the context of the preferred group of transcription factors as shown in row 1 of Table 2, the transcription factors NKX2-5 and HAND2 can be replaced with HAND1. In other words, if it is not possible to increase the protein expression or amount of any of the given transcription factors NKX2.5 or HAND2 (for example, if there is no nucleic acid construct available for increasing expression directly), then it is possible to seek to increase the expression of HAND1, instead.

The transcription factors and other protein factors referred to herein are referred to by the HUGO Gene Nomenclature Committee (HGNC) Symbol. Table 3 provides exemplary Ensemble Gene ID and Uniprot IDs for the transcription factors recited herein. The nucleotide sequences are derived from the Ensembl database (Flicek et al. (2014). Nucleic Acids Research Volume 42, Issue Dl. Pp. D749-D755) version 83. Also contemplated for use in the invention is any homolog, ortholog or paralog of a transcription factor referred to herein.

The skilled person will appreciate that this information may be used in performing the methods of the present invention, for example, for the purposes of providing increased amounts of transcription factors in source cells, or providing nucleic acids or the like for recombinantly expressing a transcription factor in a source cell.

TABLE 3
Accession numbers identifying nucleotide sequences and amino
acid sequences of transcription factors referred to herein.

| Transcription factor | | |
|---|---|---|
| Associated Gene Name | Ensembl Gene ID | Uniprot ID |
| ABRA | ENSG00000174429 | Q8N0Z2 |
| AEBP1 | ENSG00000106624 | Q8IUX7 |
| AHR | ENSG00000106546 | P35869 |
| ANKRD1 | ENSG00000148677 | Q15327 |
| ANKRD1 | ENSG00000148677 | Q15327 |
| ARNT2 | ENSG00000172379 | Q9HBZ2 |
| ATOH8 | ENSG00000168874 | Q96SQ7 |
| BACH2 | ENSG00000112182 | Q9BYV9 |
| BARX1 | ENSG00000131668 | Q9HBU1 |
| BMP10 | ENSG00000163217 | O95393 |
| BMP4 | ENSG00000125378 | P12644 |
| CDH1 | ENSG00000039068 | P12830 |
| CDH1 | ENSG00000039068 | P12830 |
| CEBPB | ENSG00000172216 | P17676 |
| CREB3 | ENSG00000107175 | I43889 |
| CUX1 | ENSG00000257923 | P39880 |
| DBP | ENSG00000105516 | Q10586 |
| DLX3 | ENSG00000064195 | O60479 |
| DLX5 | ENSG00000105880 | P56178 |
| E2F1 | ENSG00000101412 | Q01094 |
| E2F5 | ENSG00000133740 | Q15329 |
| E2F8 | ENSG00000129173 | A0AVK6 |
| EBF1 | ENSG00000164330 | Q9UH73 |
| ELF4 | ENSG00000102034 | Q99607 |
| ENPP2 | ENSG00000136960 | Q13822 |
| EPAS1 | ENSG00000116016 | Q99814 |
| ESRRA | ENSG00000173153 | P11474 |
| FGF2 | ENSG00000138685 | P09038 |
| FHL2 | ENSG00000115641 | Q14192 |
| FOS | ENSG00000170345 | P01100 |
| FOSB | ENSG00000125740 | P53539 |
| FOSL1 | ENSG00000175592 | P15407 |
| FOSL2 | ENSG00000075426 | P15408 |
| FOSL2 | ENSG00000075426 | P15408 |
| FOXA2 | ENSG00000125798 | Q9Y261 |
| FOXC1 | ENSG00000054598 | Q12948 |
| FOXC2 | ENSG00000176692 | Q99958 |
| FOXD1 | ENSG00000251493 | Q16676 |
| FOXQ1 | ENSG00000164379 | Q9C009 |
| GATA1 | ENSG00000102145 | P15976 |
| GATA4 | ENSG00000136574 | P43694 |
| GATA6 | ENSG00000141448 | Q92908 |
| GDF6 | ENSG00000156466 | Q6KF10 |
| GFI1 | ENSG00000162676 | Q99684 |
| GFI16 | ENSG00000165702 | Q5VTD9 |
| HAND1 | ENSG00000113196 | O96004 |
| HAND2 | ENSG00000164107 | P61296 |
| HES1 | ENSG00000114315 | Q14469 |
| HIC1 | ENSG00000177374 | Q14526 |
| HMGB2 | ENSG00000164104 | P26583 |
| HMOX1 | ENSG00000100292 | P09601 |
| HOXA11 | ENSG00000005073 | P31270 |
| HOXA7 | ENSG00000122592 | P31268 |
| HOXA9 | ENSG00000078399 | P31269 |
| HOX67 | ENSG00000260027 | P09629 |
| HOXC10 | ENSG00000180818 | Q9NYD6 |
| HR | ENSG00000168453 | O43593 |
| ICAM1 | ENSG00000090339 | P05362 |
| ID1 | ENSG00000125968 | P41134 |
| ID4 | ENSG00000172201 | P47928 |
| IGF1 | ENSG00000017427 | P05019 |
| IGF2 | ENSG00000167244 | P01344 |
| IL1B | ENSG00000125538 | P01584 |
| IL6 | ENSG00000136244 | P05231 |
| INHBA | ENSG00000122641 | P08476 |
| IRF1 | ENSG00000125347 | P10914 |
| IRX5 | ENSG00000176842 | P78411 |
| JUN | ENSG00000177606 | P05412 |
| JUNB | ENSG00000171223 | P17275 |
| KLF1 | ENSG00000105610 | Q13351 |
| KLHL31 | ENSG00000124743 | Q9H511 |
| LBH | ENSG00000213626 | Q53QV2 |
| LEF1 | ENSG00000138795 | Q9UJU2 |
| LIF | ENSG00000128342 | P15018 |
| LMO3 | ENSG00000048540 | Q8TAP4 |
| LMO4 | ENSG00000143013 | P61968 |
| MAFB | ENSG00000204103 | Q9Y5Q3 |
| MEIS1 | ENSG00000143995 | O00470 |
| MEOX2 | ENSG00000106511 | P50222 |
| MSX1 | ENSG00000163132 | P28360 |
| MXD4 | ENSG00000123933 | Q14582 |
| MYB | ENSG00000118513 | P10242 |
| MYBL1 | ENSG00000185697 | P10243 |
| MYBL2 | ENSG00000101057 | P10244 |
| MYC | ENSG00000136997 | P01106 |
| MYOCD | ENSG00000141052 | Q8IZQ8 |
| MYOD1 | ENSG00000129152 | P15172 |
| MYOG | ENSG00000122180 | P15173 |
| NFATC2 | ENSG00000101096 | Q13469 |
| NFATC4 | ENSG00000100968 | Q14934 |
| NFE2 | ENSG00000123405 | Q16621 |
| NFIX | ENSG00000008441 | Q14938 |
| NFKB1 | ENSG00000109320 | P19838 |
| NFKBIA | ENSG00000100906 | P25963 |
| NFKBIZ | ENSG00000144802 | Q9BYH8 |
| NKX2-1 | ENSG00000136352 | P43699 |
| NKX2-5 | ENSG00000183072 | P52952 |
| NOTCH1 | ENSG00000148400 | P46531 |
| NOTCH3 | ENSG00000074181 | Q9UM47 |
| NR2F2 | ENSG00000185551 | P24468 |
| NR3C1 | ENSG00000113580 | P04150 |
| OTX1 | ENSG00000115507 | P32242 |
| PAX6 | ENSG00000007372 | P26367 |
| PBX1 | ENSG00000185630 | P40424 |
| PITX1 | ENSG00000069011 | P78337 |
| PITX3 | ENSG00000107859 | O75364 |
| PKNOX2 | ENSG00000165495 | Q96KN3 |
| POU3F2 | ENSG00000184486 | P20265 |
| PPARGC1A | ENSG00000109819 | Q9UBK2 |
| PRRX1 | ENSG00000116132 | P54821 |
| PRRX2 | ENSG00000167157 | Q99811 |
| RARB | ENSG00000077092 | P10826 |
| RARG | ENSG00000172819 | P13631 |
| RCAN1 | ENSG00000159200 | P53805 |
| REL | ENSG00000162924 | Q04864 |
| RIPK3 | ENSG00000129465 | Q9Y572 |
| RORA | ENSG00000069667 | P35398 |
| RUNX1 | ENSG00000159216 | Q01196 |
| RUNX1T1 | ENSG00000079102 | Q06455 |
| RUNX2 | ENSG00000124813 | Q13950 |
| RUNX3 | ENSG00000020633 | Q13761 |
| S100A1 | ENSG00000160678 | P23297 |
| SIX1 | ENSG00000126778 | Q15475 |
| SIX2 | ENSG00000170577 | Q9NPC8 |
| SIX5 | ENSG00000177045 | Q8N196 |
| SMAD1 | ENSG00000170365 | Q15797 |
| SMAD6 | ENSG00000137834 | O43541 |
| SMAD7 | ENSG00000101665 | O15105 |
| SMAD9 | ENSG00000120693 | O15198 |
| SMYD1 | ENSG00000115593 | Q8NB12 |
| SNAI2 | ENSG00000019549 | O43623 |
| SOX17 | ENSG00000164736 | Q9H6I2 |
| SOX2 | ENSG00000181449 | P48431 |
| SOX5 | ENSG00000134532 | P35711 |
| SOX8 | ENSG00000005513 | P57073 |
| SOX9 | ENSG00000125398 | P48436 |
| SREBF1 | ENSG00000072310 | P36956 |
| TAL1 | ENSG00000162367 | P17542 |
| TBX20 | ENSG00000164532 | Q9UMR3 |
| TBX5 | ENSG00000089225 | Q99593 |
| TCF21 | ENSG00000118526 | O43680 |
| TCF7L1 | ENSG00000152284 | Q9HCS4 |
| TFAP2A | ENSG00000137203 | P05549 |
| TGFB3 | ENSG00000119699 | P10600 |
| TP63 | ENSG00000073282 | Q9H3D4 |
| TRPS1 | ENSG00000104447 | Q9UHF7 |
| TSC22D1 | ENSG00000102804 | Q15714 |
| TWIST1 | ENSG00000122691 | Q15672 |
| VDR | ENSG00000111424 | P11473 |
| VEGFA | ENSG00000112715 | P15692 |
| VGLL4 | ENSG00000144560 | Q14135 |
| WWTR1 | ENSG00000018408 | Q9GZV5 |
| ZEB2 | ENSG00000169554 | O60315 |
| ZFP42 | ENSG00000179059 | Q96MM3 |
| ZIC1 | ENSG00000152977 | Q15915 |

The term a “variant” in referring to a polypeptide that is at least 70%, 80%, 85%, 90%, 95%, 98%, or 99% identical to the full length polypeptide. The present invention contemplates the use of variants of the transcription factors described herein, including variants of the TFs listed in Tables 1 and 2 and the sequences listed in Table 3. The variant could be a fragment of full length polypeptide or a naturally occurring splice variant. The variant could be a polypeptide at least 70%, 80%, 85%, 90%, 95%, 98%, or 99% identical to a fragment of the polypeptide, wherein the fragment is at least 50%, 60%, 70%, 80%, 85%, 90%, 95%, 98%, or 99% A as long as the full length wild type polypeptide or a domain thereof has a functional activity of interest such as the ability to promote conversion of a source cell type to a target cell type. In some embodiments the domain is at least 100, 200, 300, or 400 amino acids in length, beginning at any amino acid position in the sequence and extending toward the C-terminus. Variations known in the art to eliminate or substantially reduce the activity of the protein are preferably avoided. In some embodiments, the variant lacks an N- and/or C-terminal portion of the full length polypeptide, e.g., up to 10, 20, or 50 amino acids from either terminus is lacking. In some embodiments the polypeptide has the sequence of a mature (full length) polypeptide, by which is meant a polypeptide that has had one or more portions such as a signal peptide removed during normal intracellular proteolytic processing (e.g., during co-translational or post-translational processing). In some embodiments wherein the protein is produced other than by purifying it from cells that naturally express it, the protein is a chimeric polypeptide, by which is meant that it contains portions from two or more different species. In some embodiments wherein a protein is produced other than by purifying it from cells that naturally express it, the protein is a derivative, by which is meant that the protein comprises additional sequences not related to the protein so long as those sequences do not substantially reduce the biological activity of the protein. One of skill in the art will be aware of, or will readily be able to ascertain, whether a particular polypeptide variant, fragment, or derivative is functional using assays known in the art. For example, the ability of a variant of a transcription factor to convert a source cell to a target cell type can be assessed using the assays as disclose herein in the Examples. Other convenient assays include measuring the ability to activate transcription of a reporter construct containing a transcription factor binding site operably linked to a nucleic acid sequence encoding a detectable marker such as luciferase. In certain embodiments of the invention a functional variant or fragment has at least 50%, 60%, 70%, 80%, 90%, 95% or more of the activity of the full length wild type polypeptide.

The term “increasing the amount of” with respect to increasing an amount of a transcription factor, refers to increasing the quantity of the transcription factor in a cell of interest (e.g., a source cell such as a fibroblast). In some embodiments, the amount of transcription factor is “increased” in a cell of interest (e.g., a cell into which an expression cassette directing expression of a polynucleotide encoding one or more transcription factors has been introduced) when the quantity of transcription factor is at least 10%, 20%, 30%, 40%, 50%, 60%, 70%, 80%, 90%, or more relative to a control (e.g., a fibroblast into which none of said expression cassettes have been introduced). However, any method of increasing an amount of a transcription factor is contemplated including any method that increases the amount, rate or efficiency of transcription, translation, stability or activity of a transcription factor (or the pre-mRNA or mRNA encoding it). In addition, down-regulation or interference of a negative regulator of transcription expression, increasing efficiency of existing translation (e.g. SINEUP) are also considered.

The term “agent” as used herein means any compound or substance such as, but not limited to, a small molecule, nucleic acid, polypeptide, peptide, drug, ion, etc. An “agent” can be any chemical, entity or moiety, including without limitation synthetic and naturally-occurring proteinaceous and non-proteinaceous entities. In some embodiments, an agent is nucleic acid, nucleic acid analogues, proteins, antibodies, peptides, aptamers, oligomer of nucleic acids, amino acids, or carbohydrates including without limitation proteins, oligonucleotides, ribozymes, DNAzymes, glycoproteins, siRNAs, lipoproteins, aptamers, and modifications and combinations thereof etc. In certain embodiments, agents are small molecule having a chemical moiety. For example, chemical moieties included unsubstituted or substituted alkyl, aromatic, or heterocyclic moieties including macrolides, leptomycins and related natural products or analogues thereof. Compounds can be known to have a desired activity and/or property, or can be selected from a library of diverse compounds.

The term “exogenous,” when used in relation to a protein, gene, nucleic acid, or polynucleotide in a cell or organism refers to a protein, gene, nucleic acid, or polynucleotide that has been introduced into the cell or organism by artificial or natural means; or in relation to a cell, refers to a cell that was isolated and subsequently introduced to other cells or to an organism by artificial or natural means. An exogenous nucleic acid may be from a different organism or cell, or it may be one or more additional copies of a nucleic acid that occurs naturally within the organism or cell. An exogenous cell may be from a different organism, or it may be from the same organism. By way of a non-limiting example, an exogenous nucleic acid is one that is in a chromosomal location different from that of natural cells, or is otherwise flanked by a different nucleic acid sequence than that found in nature. An exogenous nucleic acid may also be extra-chromosomal, such as an episomal vector.

Screening one or more candidate agents for the ability to increase the amount of the one or more transcription factors required for conversion of a source cell type to a target cell type may include the steps of contacting a system that allows the product or expression of a transcription factor with the candidate agent and determining whether the amount of the transcription factor has increased. The system may be in vivo, for example a tissue or cell in an organism, or in vitro, a cell isolated from an organism or an in vitro transcription assay, or ex vivo in a cell or tissue. The amount of transcription factor may be measured directly or indirectly, and either by determining the amount of protein or RNA (e.g. mRNA or pre-mRNA). The candidate agent function to increase the amount of a transcription factor by increasing any step in the transcription of the gene encoding the transcription factor or increase the translation of corresponding mRNA. Alternatively, the candidate agent may decrease the inhibitory activity of a repressor of transcription of the gene encoding the transcription factor or the activity of a molecule that causes the degradation of the mRNA encoding the transcription factor or the protein of the transcription factor itself.

Suitable detection means include the use of labels such as radionucleotides, enzymes, coenzymes, fluorescers, chemiluminescers, chromogens, enzyme substrates or co-factors, enzyme inhibitors, prosthetic group complexes, free radicals, particles, dyes, and the like. Such labelled reagents may be used in a variety of well-known assays, such as radioimmunoassays, enzyme immunoassays, e.g., ELISA, fluorescent immunoassays, and the like. See, for example, U.S. Pat. Nos. 3,766,162; 3,791,932; 3,817,837; and 4,233,402.

The methods of the invention include high-throughput screening applications. For example, a high-throughput screening assay may be used which comprises any of the assays according to the invention wherein aliquots of a system that allows the product or expression of a transcription factor are exposed to a plurality of candidate agents within different wells of a multi-well plate. Further, a high-throughput screening assay according to the disclosure involves aliquots of a system that allows the product or expression of a transcription factor which are exposed to a plurality of candidate agents in a miniaturized assay system of any kind.

The method of the disclosure may be “miniaturized” in an assay system through any acceptable method of miniaturization, including but not limited to multi-well plates, such as 24, 48, 96 or 384-wells per plate, microchips or slides. The assay may be reduced in size to be conducted on a micro-chip support, advantageously involving smaller amounts of reagent and other materials. Any miniaturization of the process which is conducive to high-throughput screening is within the scope of the invention.

In any method of the invention the target cells can be transferred into the same mammal from which the source cells were obtained. In other words, the source cells used in a method of the invention can be an autologous cell, i.e., can be obtained from the same individual in which the target cells are to be administered. Alternatively, the target cell can be allogenically transferred into another individual. Preferably, the cell is autologous to the subject in a method of treating or preventing a medical condition in the individual.

The term “cell culture medium” (also referred to herein as a “culture medium” or “medium”) as referred to herein is a medium for culturing cells containing nutrients that maintain cell viability and support proliferation. The cell culture medium may contain any of the following in an appropriate combination: salt(s), buffer(s), amino acids, glucose or other sugar(s), antibiotics, serum or serum replacement, and other components such as peptide growth factors, etc. Cell culture media ordinarily used for particular cell types are known to those skilled in the art. Exemplary cell culture medium for use in methods of the invention are shown in Table 4.

TABLE 4
Cell culture media that can be used to culture cell types

| Cell | Media | Cat#: | Company |
|---|---|---|---|
| MSCs of adipose | StemPro ® Human | R7788-110 | ThermoFisher |
| | Adipose-Derived | | |
| | Stem Cell Kit | | |
| MSCs of bone | StemPro ® BM | A15652 | ThermoFisher |
| marrow | Mesenchymal Stem | | Life |
| | Cells kit | | Technologies |
| | Alpha-MEM with 15% | | |
| | FBS, glutamine, | | |
| | penicillin ands | | |
| | treptomycin | | |
| Cardiac fibroblasts | DMEM (high glucose, | N/A | ThermoFisher |
| | without sodium | | Life |
| | pyruvate), 10% FBS, | | Technologies |
| | 1% | | |
| | penicillin/streptomycin | | |
| Cardiomyocytes | DMEM/medium 199 | N/A | ThermoFisher |
| | (4:1) with 10% FBS | | Life |
| | | | Technologies |
| | | | Sigma Aldrich |

A nucleic acid or vector comprising a nucleic acid as described herein may include one or more of the sequences referred to above in Table 3 or a sequence encoding any one or more of the transcription factors listed in Tables 1 and 2.

The term “expression” refers to the cellular processes involved in producing RNA and proteins and as appropriate, secreting proteins, including where applicable, but not limited to, for example, transcription, translation, folding, modification and processing.

The term “isolated” or “partially purified” as used herein refers, in the case of a nucleic acid or polypeptide, to a nucleic acid or polypeptide separated from at least one other component (e.g., nucleic acid or polypeptide) that is present with the nucleic acid or polypeptide as found in its natural source and/or that would be present with the nucleic acid or polypeptide when expressed by a cell, or secreted in the case of secreted polypeptides. A chemically synthesized nucleic acid or polypeptide or one synthesized using in vitro transcription/translation is considered “isolated”.

The term “vector” refers to a carrier DNA molecule into which a DNA sequence can be inserted for introduction into a host or source cell. Preferred vectors are those capable of autonomous replication and/or expression of nucleic acids to which they are linked. Vectors capable of directing the expression of genes to which they are operatively linked are referred to herein as “expression vectors”. Thus, an “expression vector” is a specialized vector that contains the necessary regulatory regions needed for expression of a gene of interest in a host cell. In some embodiments the gene of interest is operably linked to another sequence in the vector. Vectors can be viral vectors or non-viral vectors. Should viral vectors be used, it is preferred the viral vectors are replication defective, which can be achieved for example by removing all viral nucleic acids that encode for replication. A replication defective viral vector will still retain its infective properties and enters the cells in a similar manner as a replicating adenoviral vector, however once admitted to the cell a replication defective viral vector does not reproduce or multiply. Vectors also encompass liposomes and nanoparticles and other means to deliver DNA molecule to a cell.

The term “operably linked” means that the regulatory sequences necessary for expression of the coding sequence are placed in the DNA molecule in the appropriate positions relative to the coding sequence so as to effect expression of the coding sequence. This same definition is sometimes applied to the arrangement of coding sequences and transcription control elements (e.g. promoters, enhancers, and termination elements) in an expression vector. The term “operatively linked” includes having an appropriate start signal (e.g. ATG) in front of the polynucleotide sequence to be expressed, and maintaining the correct reading frame to permit expression of the polynucleotide sequence under the control of the expression control sequence, and production of the desired polypeptide encoded by the polynucleotide sequence.

The term “viral vectors” refers to the use of viruses, or virus-associated vectors as carriers of a nucleic acid construct into a cell. Constructs may be integrated and packaged into non-replicating, defective viral genomes like Adenovirus, Adeno-associated virus (AAV), or Herpes simplex virus (HSV) or others, including reteroviral and lentiviral vectors, for infection or transduction into cells. The vector may or may not be incorporated into the cell's genome. The constructs may include viral sequences for transfection, if desired. Alternatively, the construct may be incorporated into vectors capable of episomal replication, e.g EPV and EBV vectors.

As used herein, the term “adenovirus” refers to a virus of the family Adenovirida. Adenoviruses are medium-sized (90-100 nm), nonenveloped (naked) icosahedral viruses composed of a nucleocapsid and a double-stranded linear DNA genome.

As used herein, the term “non-integrating viral vector” refers to a viral vector that does not integrate into the host genome; the expression of the gene delivered by the viral vector is temporary. Since there is little to no integration into the host genome, non-integrating viral vectors have the advantage of not producing DNA mutations by inserting at a random point in the genome. For example, a non-integrating viral vector remains extra-chromosomal and does not insert its genes into the host genome, potentially disrupting the expression of endogenous genes. Non-integrating viral vectors can include, but are not limited to, the following: adenovirus, alphavirus, picornavirus, and vaccinia virus. These viral vectors are “non-integrating” viral vectors as the term is used herein, despite the possibility that any of them may, in some rare circumstances, integrate viral nucleic acid into a host cell's genome. What is critical is that the viral vectors used in the methods described herein do not, as a rule or as a primary part of their life cycle under the conditions employed, integrate their nucleic acid into a host cell's genome.

The vectors described herein can be constructed and engineered using methods generally known in the scientific literature to increase their safety for use in therapy, to include selection and enrichment markers, if desired, and to optimize expression of nucleotide sequences contained thereon. The vectors should include structural components that permit the vector to self-replicate in the source cell type. For example, the known Epstein Barr oriP/Nuclear Antigen-1 (EBNA-I) combination (see, e.g., Lindner, S. E. and B. Sugden, The plasmid replicon of Epstein-Barr virus: mechanistic insights into efficient, licensed, extrachromosomal replication in human cells, Plasmid 58:1 (2007), incorporated by reference as if set forth herein in its entirety) is sufficient to support vector self-replication and other combinations known to function in mammalian, particularly primate, cells can also be employed. Standard techniques for the construction of expression vectors suitable for use in the present invention are well-known to one of ordinary skill in the art and can be found in publications such as Sambrook J, et al., “Molecular cloning: a laboratory manual,” (3rd ed. Cold Spring harbor Press, Cold Spring Harbor, N.Y. 2001), incorporated herein by reference as if set forth in its entirety.

In the methods of the invention, genetic material encoding the relevant transcription factors required for a conversion is delivered into the source cells via one or more reprogramming vectors. Each transcription factor can be introduced into the source cells as a polynucleotide transgene that encodes the transcription factor operably linked to a heterologous promoter that can drive expression of the polynucleotide in the source cell.

Suitable reprogramming vectors are any described herein, including episomal vectors, such as plasmids, that do not encode all or part of a viral genome sufficient to give rise to an infectious or replication-competent virus, although the vectors can contain structural elements obtained from one or more virus. One or a plurality of reprogramming vectors can be introduced into a single source cell. One or more transgenes can be provided on a single reprogramming vector. One strong, constitutive transcriptional promoter can provide transcriptional control for a plurality of transgenes, which can be provided as an expression cassette. Separate expression cassettes on a vector can be under the transcriptional control of separate strong, constitutive promoters, which can be copies of the same promoter or can be distinct promoters. Various heterologous promoters are known in the art and can be used depending on factors such as the desired expression level of the transcription factor. It can be advantageous, as exemplified below, to control transcription of separate expression cassettes using distinct promoters having distinct strengths in the source cells. Another consideration in selection of the transcriptional promoters is the rate at which the promoter(s) is silenced. The skilled artisan will appreciate that it can be advantageous to reduce expression of one or more transgenes or transgene expression cassettes after the product of the gene(s) has completed or substantially completed its role in the reprogramming method. Exemplary promoters are the human EF1α elongation factor promoter, CMV cytomegalovirus immediate early promoter and CAG chicken albumin promoter, and corresponding homologous promoters from other species. In human somatic cells, both EF1α and CMV are strong promoters, but the CMV promoter is silenced more efficiently than the EF1α promoter such that expression of transgenes under control of the former is turned off sooner than that of transgenes under control of the latter. The transcription factors can be expressed in the source cells in a relative ratio that can be varied to modulate reprogramming efficiency. Preferably, where a plurality of transgenes is encoded on a single transcript, an internal ribosome entry site is provided upstream of transgene(s) distal from the transcriptional promoter. Although the relative ratio of factors can vary depending upon the factors delivered, one of ordinary skill in possession of this disclosure can determine an optimal ratio of factors.

The skilled artisan will appreciate that the advantageous efficiency of introducing all factors via a single vector rather than via a plurality of vectors, but that as total vector size increases, it becomes increasingly difficult to introduce the vector. The skilled artisan will also appreciate that position of a transcription factor on a vector can affect its temporal expression, and the resulting reprogramming efficiency. As such, Applicants employed various combinations of factors on combinations of vectors. Several such combinations are here shown to support reprogramming.

After introduction of the reprogramming vector(s) and while the source cells are being reprogrammed, the vectors can persist in target cells while the introduced transgenes are transcribed and translated. Transgene expression can be advantageously downregulated or turned off in cells that have been reprogrammed to a target cell type. The reprogramming vector(s) can remain extra-chromosomal. At extremely low efficiency, the vector(s) can integrate into the cells' genome. The examples that follow are intended to illustrate but in no way limit the present invention.

Suitable methods for nucleic acid delivery for transformation of a cell, a tissue or an organism for use with the current invention are believed to include virtually any method by which a nucleic acid (e.g., DNA) can be introduced into a cell, a tissue or an organism, as described herein or as would be known to one of ordinary skill in the art (e.g., Stadtfeld and Hochedlinger, Nature Methods 6(5):329-330 (2009); Yusa et al., Nat. Methods 6:363-369 (2009); Woltjen, et al., Nature 458, 766-770 (9 Apr. 2009)). Such methods include, but are not limited to, direct delivery of DNA such as by ex vivo transfection (Wilson et al., Science, 244:1344-1346, 1989, Nabel and Baltimore, Nature 326:711-713, 1987), optionally with a lipid-based transfection reagent such as Fugene6 (Roche) or Lipofectamine (Invitrogen), by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, J. Cell Biol., 101:1094-1099, 1985; U.S. Pat. No. 5,789,215, incorporated herein by reference); by electroporation (U.S. Pat. No. 5,384,253, incorporated herein by reference; Tur-Kaspa et al., Mol. Cell Biol., 6:716-718, 1986; Potter et al., Proc. Nat'l Acad. Sci. USA, 81:7161-7165, 1984); by calcium phosphate precipitation (Graham and Van Der Eb, Virology, 52:456-467, 1973; Chen and Okayama, Mol. Cell Biol., 7(8):2745-2752, 1987; Rippe et al., Mol. Cell Biol., 10:689-695, 1990); by using DEAE-dextran followed by polyethylene glycol (Gopal, Mol. Cell Biol., 5:1188-1190, 1985); by direct sonic loading (Fechheimer et al., Proc. Nat'l Acad. Sci. USA, 84:8463-8467, 1987); by liposome mediated transfection (Nicolau and Sene, Biochim. Biophys. Acta, 721:185-190, 1982; Fraley et al., Proc. Nat'l Acad. Sci. USA, 76:3348-3352, 1979; Nicolau et al., Methods Enzymol., 149:157-176, 1987; Wong et al., Gene, 10:87-94, 1980; Kaneda et al., Science, 243:375-378, 1989; Kato et al., J Biol. Chem., 266:3361-3364, 1991) and receptor-mediated transfection (Wu and Wu, Biochemistry, 27:887-892, 1988; Wu and Wu, J. Biol. Chem., 262:4429-4432, 1987); and any combination of such methods, each of which is incorporated herein by reference.

A number of polypeptides capable of mediating introduction of associated molecules into a cell have been described previously and can be adapted to the present invention. See, e.g., Langel (2002) Cell Penetrating Peptides: Processes and Applications, CRC Press, Pharmacology and Toxicology Series. Examples of polypeptide sequences that enhance transport across membranes include, but are not limited to, the Drosophila homeoprotein antennapedia transcription protein (AntHD) (Joliot et al., New Biol. 3: 1121-34, 1991; Joliot et al., Proc. Natl. Acad. Sci. USA, 88: 1864-8, 1991; Le Roux et al., Proc. Natl. Acad. Sci. USA, 90: 9120-4, 1993), the herpes simplex virus structural protein VP22 (Elliott and O'Hare, Cell 88: 223-33, 1997); the HIV-1 transcriptional activator TAT protein (Green and Loewenstein, Cell 55: 1179-1188, 1988; Frankel and Pabo, Cell 55: 1 289-1193, 1988); Kaposi FGF signal sequence (kFGF); protein transduction domain-4 (PTD4); Penetratin, M918, Transportan-10; a nuclear localization sequence, a PEP-I peptide; an amphipathic peptide (e.g., an MPG peptide); delivery enhancing transporters such as described in U.S. Pat. No. 6,730,293 (including but not limited to an peptide sequence comprising at least 5-25 or more contiguous arginines or 5-25 or more arginines in a contiguous set of 30, 40, or 50 amino acids; including but not limited to an peptide having sufficient, e.g., at least 5, guanidino or amidino moieties); and commercially available Penetratin™ 1 peptide, and the Diatos Peptide Vectors (“DPVs”) of the Vectocell® platform available from Daitos S. A. of Paris, France. See also, WO/2005/084158 and WO/2007/123667 and additional transporters described therein. Not only can these proteins pass through the plasma membrane but the attachment of other proteins, such as the transcription factors described herein, is sufficient to stimulate the cellular uptake of these complexes.

The present invention includes the following non-limiting Examples.

### EXAMPLES

### Example 1

Candidate transcription factors for conversion of cardiac fibroblasts to fetal cardiomyocytes were identified according to the methods previously described in Rackham et al (2016) Nature Genetics, 48(3): 331-335.

Plasmids

Nucleic acids encoding eight transcription factors, BMP10, GATA6, TBX5, ANKRD1, HAND1, PPARGC1A, NKX2.5, and GATA4 were cloned into an episomal expression vector pCEP4. This vector was transfected into cardiac fibroblasts for the purpose of transiently expressing these transcription factors in the fibroblasts to determine whether it is possible to reprogram the cells in to fetal cardiomyocytes.

Transfection

In the data presented below, transfection was carried out by nucleofection using an Amaxa 4D-Nucleofectror (P2 Primary cell kit from Lonza, Program FF-135).

### Example 2

Single Factor Expression

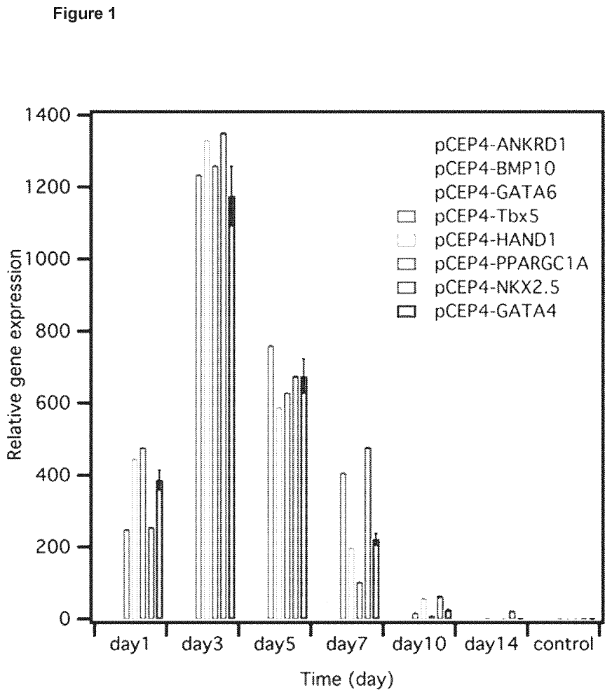

Human fetal cardiac fibroblast (HFCFs, P6) were transfected with non-integrating plasmids containing a transcription factor by nucleofection, in which qPCR was performed to quantify the expression level of each factors as a function of time. Relative changes in gene expression were calculated using 2^{-ΔΔCt} method with GPDH as a reference gene. The data confirms that the episomal plasmids are indeed able to effect temporal expression of the genes and that there expression was effectively suppressed after day 14 (FIG. 1).

### Example 3

Reprogramming Human Fetal Cardiac Fibroblasts (hFCFs)

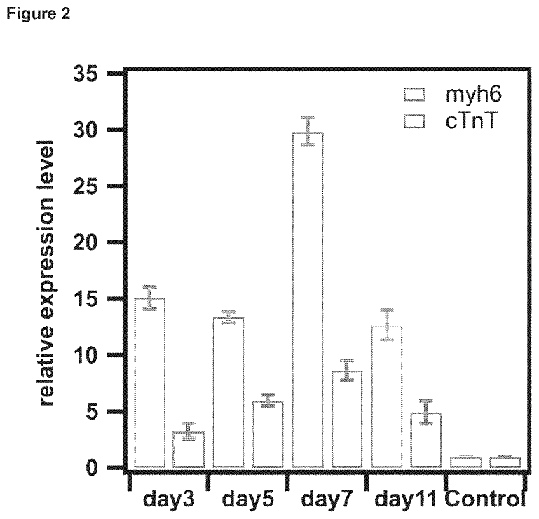

In the first set of experiments using the episomal vectors carrying the factors, we attempted reprogramming of hFCFs using nucleofection with the 8 transcription factors identified in Example 1, and the expression of endogenous cardiac markers, including myh6, and cTnT, were quantified by qPCR (on total unsorted cells) up to day 11 (FIG. 2). Note that the decrease in the expression levels of the cardiac genes is expected after day 7 due to the culture being overgrown by fibroblasts that were not transfected and thus not transitioning. This results in the total RNA from the population being diluted with normal fibroblasts.

### Example 4

Reprogramming of Reporter Cells Line, hFCFs Carrying the NCX-1-GFP Reporter

Following these first set of experiments using the hFCFs, a second round of experiments was pursued using an in-house generated hFCFs that have been stably transduced with NCX-1-GFP. These cells express GFP protein when the NCX-1 gene is turned on. The NCX-1 protein is critical in the functional behaviour of cardiomyocytes, as it enables them to beat. We have previously shown that this reporter was highly effective at highlighting cells that were transitioning from embryonic stem cells or induced pluripotent stem cells to a cardiomyocyte fate. We have also shown that the cells within a population of stem cells that expressed GFP (indicating their expression of NCX-1 gene) were also 100% correlated with those that then turned into beating cardiomyocytes—that is, it is a robust reporter of function.

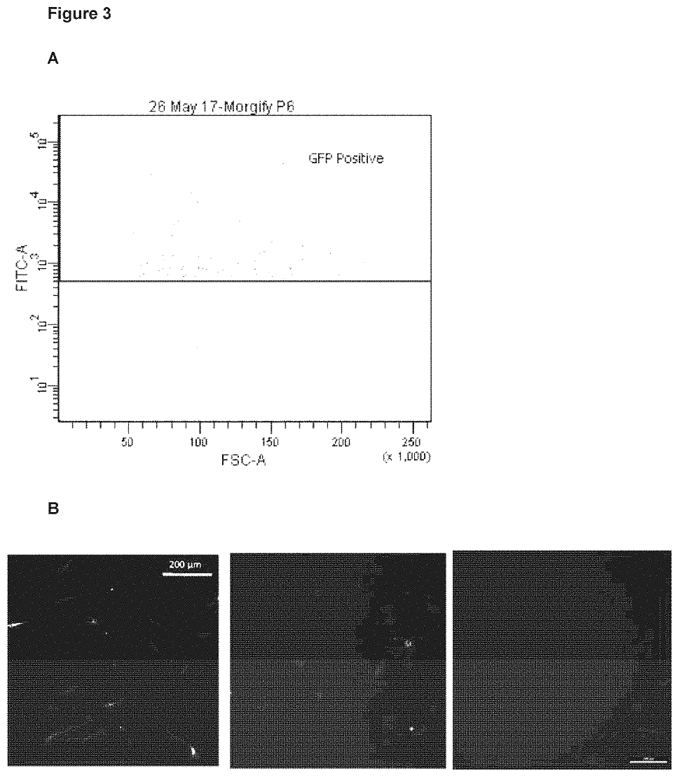

FIG. 3 shows a FACS plot that confirmed at day 8, 3.7% of human cells were expressing GFP (NCX-1). The second figure shows these cells at day 8 and 14 (taken under a fluorescent microscope): LHS—unsorted day 8; Middle—post sorting and reseeding day 8; RHS—expanded sorted cells at day 14.

### Example 5

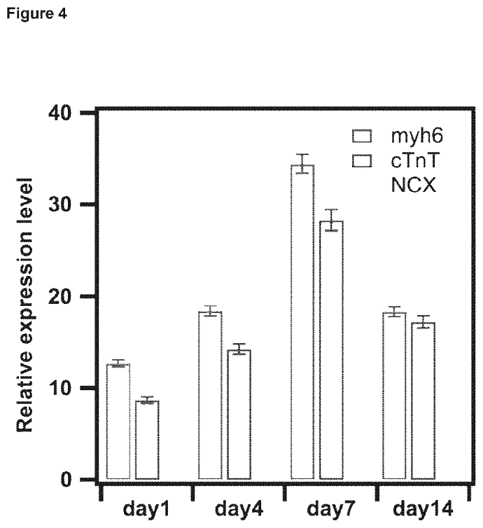

FIG. 4 shows the expression levels of cardiac genes (cTnT, aMHC, NCX-1) at up to day 14, confirming substantial upregulation and robust increases in each of these genes. Day 14 data for this panel of genes shows a decrease in myh6 and cTnT, but maintenance of NCX-1. This highlights the conversion efficiency as these levels of gene expression are for the total population of cells (i.e. not just for sorted GFP+ cells), both non-GFP and GFP+ cells, of which the GFP+ cells are the smaller population within the total by day 14.

### Example 6

Reprogramming of Reporter Cells Line, hFCFs Carrying the cTnT-EGFP Reporter

Human fetal cardiac fibroblasts (HFCFs, P3) were transfected by nucleofection with episomal (non-integrating) plasmids encoding the transcription factors GATA4, GATA6, HAND1, ANKRD1, NKX2.5, TBX5, PPARGC1A, and BMP10.

At day 2, the cell medium was changed to induction media. At day 14, the cells were transfected with a reporter construct for the structural protein cardiac troponin T (cTnT). Approximately 10% of the cells expressed the reporter protein at day 14.

It will be understood that the invention disclosed and defined in this specification extends to all alternative combinations of two or more of the individual features mentioned or evident from the text or drawings. All of these different combinations constitute various alternative aspects of the invention.

1.-37. (canceled)

38. A method for reprogramming a source cell to a cell that exhibits at least one characteristic of a cardiomyocyte comprising:
i) providing a source cell, or a cell population comprising a source cell;
ii) contacting said source cell with one or more agents that activate or increase the expression or amount of one or more transcription factors or variants thereof; and
iii) culturing said cell or cell population, and optionally monitoring the cell or cell population for at least one characteristic of a cardiomyocyte cell, wherein:
the transcription factors are one or more of those listed in Tables 1, 2 and 3.

39. The method of claim 38, wherein a source cell is a cardiac fibroblast.

40. The method of claim 38, wherein a source cell is a cardiac fibroblast and the protein expression or amount of one or more of the following transcription factors is increased:
(a) BMP10, GATA6, TBX5, ANKRD1, HAND2, PPARGC1A, NKX2-5 and GATA4;
(b) TBX5, GATA6, ANKRD1, HAND2, HAND1, PPARGC1A, NKX2-5 and GATA4;
(c) GATA6, ANKRD1, HAND1, PPARGC1A, S100A1, NKX2-5 and GATA4;
(d) FHL2, ANKRD1, HAND1, PPARGC1A, S100A1, MYOCD, NKX2-5 and GATA4;
(e) TBX20, ANKRD1, HAND1, HAND2, PPARGC1A, MYOCD, NKX2-5 and GATA4;
(f) ANKRD1, HAND1, HAND2, PPARGC1A, S100A1, MYOCD, NKX2-5 and GATA4;
(g) HAND2, HAND1, PPARGC1A, S100A1, MYOCD, KLHL31, NKX2-5 and GATA4;
(h) HAND1, PPARGC1A, S100A1, MYOCD, KLHL31, NKX2-5, GATA4 and TCF21;
(i) SMYD1, PPARGC1A, S100A1, ABRA, MYOCD, KLHL31, NKX2-5 and GATA4;
(j) PPARGC1A, S100A1, ABRA, MYOCD, KLHL31, NKX2-5, GATA4 and TCF21
(k) S100A1, ABRA, MYOCD, KLHL31, NKX2-5, GATA4 and TCF21;
(l) E2F8, ABRA, MYOCD, KLHL31, NKX2-5, GATA4 and TCF21;
(m) ABRA, MYOCD, KLHL31, NKX2-5, GATA4 and TCF21;
(n) MYOCD, KLHL31, NKX2-5, GATA4 and TCF21;
(o) IGF2, KLHL31, NKX2-5, GATA4 and TCF21; or
(p) KLHL31, NKX2-5, GATA4 and TCF21.

41. The method of claim 38, wherein the protein expression or amount of the following transcription factors is increased:
(a) HAND1, TBX5, GATA6, ANKRD1, HAND2, PPARGC1A, NKX2-5 and GATA4;
(b) BMP10, HAND1, GATA6, ANKRD1, HAND2, PPARGC1A, NKX2-5 and GATA4;
(c) BMP10, TBX5, FHL2, ANKRD1, HAND2, PPARGC1A, NKX2-5 and GATA4;
(d) BMP10, TBX5, GATA6, HAND1, HAND2, PPARGC1A, NKX2-5 and GATA4;
(e) BMP10, TBX5, GATA6, ANKRD1, HAND1, PPARGC1A, NKX2-5 and GATA4;
(f) BMP10, TBX5, GATA6, ANKRD1, HAND2, HAND1, NKX2-5 and GATA4;
(g) BMP10, TBX5, GATA6, ANKRD1, HAND2, PPARGC1A, HAND1 and GATA4; or
(h) BMP10, TBX5, GATA6, ANKRD1, HAND2, PPARGC1A, NKX2-5 and HAND1.

42. The method of claim 41, wherein the transcription factors are BMP10, GATA6, TBX5, ANKRD1, HAND1, PPARGC1A, NKX2.5, and GATA4.

43. The method of claim 38 wherein the method includes culturing the cardiac fibroblast for a sufficient time and under conditions to allow differentiation to a cardiomyocyte cell; thereby generating the cell exhibiting at least one characteristic of a cardiomyocyte cell from a cardiac fibroblast.

44. The method of claim 38, wherein the agent is selected from the group consisting of: a nucleic acid, a protein, an aptamer and small molecule, ribosome, RNAi agent, gRNA and peptide-nucleic acid (PNA) and analogues or variants thereof.

45. The method of claim 44, wherein the agent is at least one nucleic acid sequence encoding a transcription factor or protein.

46. The method of claim 44, wherein the at least one characteristic of a cardiomyocyte is up-regulation of any one or more cardiomyocyte markers and/or change in cell morphology and/or evidence of cell contractility.

47. The method of claim 46, wherein the markers of a cardiomyocyte cell include MEF2C, MYH6, ACTN1, CDH2, cardiac troponin T and GJA1.

48. The method of claim 46, wherein a marker for contractile cardiomyocytes cell is NCX-1.

49. The method of claim 38, wherein the cardiac fibroblast is reprogrammed to exhibit increased expression of NCX-1.

50. The method of claim 38, wherein the cardiomyocyte is a fetal cardiomyocyte.

51. The method of claim 38 wherein the cardiac fibroblast is a human cardiac fibroblast.

52. A cell exhibiting at least one characteristic of a cardiomyocyte cell produced by a method according to claim 38.

53. A population of cells, wherein at least 5% of cells exhibit at least one characteristic of a cardiomyocyte cell and those cells are produced by a method according to claim 38.

54. A cell or a population of cell produced by a method according to claim 38 exhibiting at least one characteristic of a cardiomyocyte cell, for the treatment of a disease of the heart.
